(19) United States
(12) Patent Application Publication
(10) Pub. No.: US 2010/0303890 A1
(43) Pub. Date: Dec. 2, 2010

(54) COPOLYMERS SUITABLE FOR USE IN CORNEAL BANDAGES

(75) Inventors: Guilhem Tourniaire, Berkshire (GB); Mark Bradley, Edinburgh (GB); Albert Liberski, Edinburgh (GB)

Correspondence Address: OCCHIUTI ROHLICEK & TSAO, LLP, 10 FAWCETT STREET, CAMBRIDGE, MA, 02138, US

(73) Assignees: University of Southampton, Hampshire (GB); The University Court of the University of Edinburgh, Edinburgh (GB)

(21) Appl. No.: 12/444,993

(22) PCT Filed: Oct. 12, 2007
(86) PCT No.: PCT/GB07/50633

§ 371 Date: Aug. 10, 2010

(30) Foreign Application Priority Data
Oct. 17, 2006 (GB) 0620537.1

(51) Int. Cl. A61F 2/14; A61K 35/44; A61P 27/02; C08F 226/00; C08L 33/08; A61F 13/00

(52) U.S. Cl. 424/429; 424/427; 424/93.7; 526/312; 524/555; 602/43

(57) ABSTRACT

Disclosed is a polymer having an affinity for corneal cells such that it can be used as a substrate for the growth of corneal cells, and so is suitable for use in a corneal bandage, especially in the form of a contact lens in which at least the cornea contacting surface is composed of the polymer. The substrate with an affinity for corneal cells is a copolymer comprising units -(A)- and -(B)- in which the units A are derived from aminoalkyl (alkyl)acrylates and the units B are derived from aryloxy, alkoxy or hydroxyalkyl (alkyl)acrylates, and (alkyl)acrylic acids.

### FIELD OF THE INVENTION

This invention concerns corneal bandages and acrylate copolymers having an affinity to corneal cells and so suitable for use in corneal bandages.

### BACKGROUND TO THE INVENTION

The paper ‘Long-term restoration of damaged corneal surfaces with autologous cultivated corneal epithelium’ by Pellegrini et al in The Lancet Volume 349, Issue 9057, 5 Apr. 1997, Pages 990-993 discloses treatment of damaged corneal surfaces using a sheet of cultivated epithelial cells mounted on a soft contact lens.

In ‘Tissue Engineering’, Volume 11, No. 11/12, 2005, pages 1710-1717, a paper by Xiaojie Hu et al entitled “Tissue Engineering of Nearly Transparent Stroma” discloses the use of polyglycolic acid fibres as a scaffold for supporting corneal cells. The cell-scaffold constructs were implanted in damaged corneas and were able to promote repair and regrowth of the corneal surface.

WO 2006/016163 (Asahi) discloses that certain polyacrylate terpolymers derived from (dialkylamino)acrylate monomers, alkoxyacrylate monomers, and other non-ionic monomers are useful in the preparation of fibrous filter pads for removing leucocytes from blood.

It is one object to provide copolymers that possess a property of binding to corneal cells, and so are suitable as substrate materials for use in corneal bandages.

It is another object of this invention to provide a corneal bandage for use as a vehicle for supplying corneal cells for corneal therapy.

### SUMMARY OF THE INVENTION

The present invention provides a polymer having an affinity for corneal cells such that it can be used as a substrate for the growth of corneal cells, and so is suitable for use in a corneal bandage, especially in the form of a contact lens in which at least the cornea contacting surface is composed of the polymer.

The substrate with an affinity for corneal cells is a copolymer comprising units -(A)- and -(B)- in which:

the units A are derived from amino alkyl (alkyl)acrylates; and
the units B are derived from aryloxy, alkoxy or hydroxyalkyl (alkyl)acrylates, and (alkyl)acrylic acids.

In one aspect, the present invention provides a corneal bandage comprising one or more bandage components in which at least the surface comprises an active copolymer having the units A and B defined above.

In one form the corneal bandage may comprise fibres or a film formed from, or grafted, or coated with, the active copolymer. In another form the corneal bandage may comprise a structure of another polymer, for example fibres or a contact lens shape, grafted or coated with the active polymer.

In a further aspect the present invention provides a method of preparing a corneal bandage for corneal therapy, comprising:

providing a contact lens with at least a surface layer of the copolymer comprising units -(A)- and -(B)-, to provide a surface having an affinity to corneal cells, and growing corneal cells on the coated surface of the contact lens to obtain a corneal cell population suitable for corneal therapy.

In another aspect the present invention provides a method of corneal therapy, comprising providing a contact lens having at least a surface of the copolymer comprising units -(A)- and -(B)-, to provide a surface having an affinity to corneal cells, growing corneal cells on the coated surface of the contact lens to obtain a corneal cell population suitable for corneal therapy, and applying the contact lens to a cornea as a corneal bandage.

### DETAILED DESCRIPTION

The present invention is concerned with corneal bandages which comprise a polymer having an affinity for corneal cells as a substrate for the growth of corneal cells.

Suitable cells for corneal therapy may be selected from corneal epithelial cells, conjunctival squamous cells and conjunctival goblet cells.

Preferably the corneal bandage is a contact lens, or polymeric construct having a contact lens shape, having at least on the cornea contacting surface a layer of a polymer having an affinity for corneal cells.

The copolymers used as a substrate with an affinity for corneal cells comprise units -(A)- and -(B)- in which:

the units A are derived from amino alkyl (alkyl)acrylates; and
the units B are derived from aryloxy, alkoxy or hydroxyalkyl (alkyl)acrylates, and (alkyl)acrylic acids.

Thus the units A may have the general formula (D)

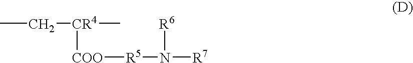

in which
R⁴, R⁶, R⁷ are independently hydrogen, lower alkyl, phenyl or substituted phenyl groups;
R⁵ is CH₂—CH₂, CHR—CH₂, CH₂—CHR^{a}, CHR^{a}—CH₂, CHR^{a}—CHR^{b}, CHR^{a}—CR^{b}R^{c}, CR^{a}R^{b}—CHR^{c}, CR^{a}R^{b}—R^{c}R^{d}, (CH₂)ₑ, where e=2-6, and R^{a}, R^{b}, R^{c}, R^{d} are lower alkyl groups and R^{a}, R^{b}, R^{c} and R^{d} maybe the same or different; and
the units B may have the general formula (E)

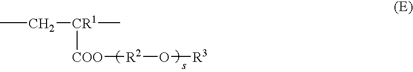

in which
R¹ is H, a lower alkyl, phenyl or substituted phenyl group;
R² is CH₂—CH₂, CH₂—CHR^{a}, CHR^{a}—CH₂, CHR^{a}—CHR^{b}, CHR^{a}—CR^{b}R^{c}, CR^{a}R^{b}—CHR^{c}, CR^{a}R^{b}—CR^{c}R^{d}, (CH₂)ₑ, where e=2-6, and R^{a}, R^{b}, R^{c}, R^{d} are lower alkyl groups and R^{a}, R^{b}, R^{c} and R^{d} maybe the same or different;
s is 0 or 1; and when s is 0, R³ is hydrogen, and when s is 1, R³ is hydrogen or a lower alkyl, phenyl or substituted phenyl group.

The copolymer suitably has the general structure

-(A)ₚ-(B)_{q}-

in which p+q=100, and 0<p, q<100. Typically 10<p, q<90.

In one favoured group of copolymers, in units B, s is 1 and R³ is hydrogen.

In another favoured group of copolymers, in units B, s is 1 and R³ is a lower alkyl group.

In a further favoured group of copolymers, in units B, s is 0 and R³ is hydrogen.

Unless indicated otherwise, references herein to “alkyl” groups means lower alkyl groups i.e. having 1-6 carbon atoms, which may be branched or linear. The group “substituted phenyl” is typically phenyl which may or may not be substituted by one or more lower alkyl groups.

The copolymer typically has the general structure

-(A)ₚ-(B)_{q}-

in which p+q=100, suitably as a random arrangement of units A and B. For example, ratios of p:q of 90:10, 10:90, 70:30, 30:70 and 50:50 may be used. Ratios of p:q in the range of 10-70:90-30 have been found especially effective.

Of the above-mentioned monomers for units A, suitable aminoalkyl (alkyl)acrylates include dialkylaminoalkyl (meth)acrylates, especially 2-(diethylamino)ethyl methacrylate, 2-(diethylamino)ethyl acrylate, 2-(dimethylamino)ethyl methacrylate, 2-(dimethylamino)ethyl acrylate, 3-(diethylamino)propyl methacrylate, 3-(diethylamino)propyl acrylate, 3-(dimethylamino)propyl methacrylate and 3-(dimethylamino)propyl acrylate.

Of the above-mentioned monomers for units A, 2-(diethylamino)ethyl methacrylate, 2-(diethylamino)ethyl acrylate, 2-(dimethylamino)ethyl methacrylate, and 2-(dimethylamino)ethyl acrylate are preferably employed from the viewpoints of availability, ease in handling in the polymerization, and the performance of the substrate.

Suitable monomers from which units B may be derived include aryloxy or alkoxy(alkyl)acrylates, especially alkoxy(meth)acrylates and alkyl(meth)acrylates.

Suitable aryl/alkoxy(meth)acrylates include 2-methoxyethyl methacrylate, 2-methoxyethyl acrylate, 2-ethoxyethyl methacrylate, 2-ethoxyethyl acrylate, 2-phenoxyethyl methacrylate, and 2-phenoxyethyl acrylate.

Of the above-mentioned monomers, 2-methoxyethyl methacrylate is preferably employed from the viewpoints of availability, ease in handling in the polymerization, and the performance as the substrate.

Other suitable monomers from which units B may be derived include: hydroxyalkyl(alkyl)acrylates, especially hydroxyalkyl(meth)acrylates.

Preferred hydroxyalkyl(meth)acrylates from the viewpoints of availability, ease in handling in the polymerization, and the performance of the substrate, include hydroxylethylmethacrylate (HEMA), hydroxypropylmethacrylate (HPMA), hydroxylbutylmethacrylate (HBMA), hydroxylethylacrylate (HEA), hydroxylpropylacrylate (HPA), and hydroxylbutylacrylate (HBA).

Further suitable monomers from which units B may be derived include (alkyl)acrylic acids, especially methacrylic and acrylic acids. Methacrylic acid is preferably employed from the viewpoints of availability, ease in handling in the polymerization, and the performance of the substrate.

In a preferred group of suitable copolymers, unit A is derived from (2-dimethylamino)ethyl methacrylate DMAEMA and unit B from methacrylic acid MAA.

In another preferred group of suitable copolymers, unit A is derived from 2-(diethylamino)ethyl methacrylate or 2-(dimethylamino)ethyl methacrylate (DEAEMA or DMAEMA) and B from a hydroxyl(ethyl or propyl)methacrylate (HEMA or HPMA).

In a further preferred group of suitable copolymers, unit A is derived from 2-diethylamino)ethyl acrylate (DEAEA) and B is derived from 2-methoxyethylmethacrylate (MEMA).

The copolymers containing units A and B preferably used in this invention may be prepared using free radical copolymerization techniques conventionally used for the preparation of polyacrylates, which typically provides random copolymers.

The efficacy of the copolymers in binding to corneal cells may be detected or confirmed by binding assays.

For use in a corneal bandage, in addition to the contact lens structure mentioned above, the copolymers may be formed into films for use as bandages. Alternatively the copolymers may be formed into fibres, or coated on fibrous supports, to form pads for use as bandages.

### Experimental

### 1. Preparation of Polymers

A mixture of AIBN, monomers and solvent was purged with nitrogen for 2 h to remove oxygen. Polymerization was left overnight under nitrogen. After reaction, the products were precipitated by the drop-wise addition of a non-polar solvent to give a solid. The polymers were re-dissolved and re-precipitated, collected and dried under vacuum at 40 C for 4 hours. Polymers were characterised by GPC, DCS and other techniques.

Using this procedure the copolymers listed in Table 1 were prepared.

TABLE 1

| co-polymers | Monomers | Monomer molar ratio |
|---|---|---|
| Example 1 | MMA/DMAEMA | 50:50 |
| Example 2 | MMA/DMAEMA | 30:70 |
| Example 3 | HEMA/DEAEMA | 50:50 |
| Example 4 | HEMA/DEAEMA | 70:30 |
| Example 5 | MEMA/DEAEA | 90:10 |
| Example 6 | HPMA/DMAEMA | 90:10 |

### 2. Affinity for Binding to Corneal Cells

Polymers were dissolved to 2% w/v in tetrahydrofuran (THF). These solutions were spin-coated onto 22 mm diameter glass coverslip using a P6708 Spincoater (Speedline Technologies, US). Following coating, the coverslips were dried under vacuum overnight at 45° C. and sterilized by exposure to UV irradiation for 15 minutes. Cover slips was sterilized before cell cultivation according to protocol described below.

1. The coverslips of 22 mm diameter were coated on one side with coatings of the copolymers of Examples 1, 4, 5 and 6. Twelve cover slips for each type of polymer were prepared.

2. The coverslips (six samples each) were placed in 12-well plates and the wells were filled with a Human Corneal Epithelial Cell Line (HCEC) medium, ‘EpiLife’, along with antibiotics (10% Penicillin/Streptomycin and Fungizone) and left for 24 h in room temperature for sterilization. Next the medium was removed and cover slips were washed once with PBS and the wells were filled with 1 ml of EpiLife with antibiotics (1% Pen/Strep and Fungizone).

Corneal epithelial cells were seeded into the wells (120000 cells/well) such that the final volume was 2 ml in each well. The plates were placed in the incubator at 37° C. for 24 h.

Controls were setup by seeding the cells into 3 wells in a 12-well plate coated with fibronectin (usually used to coat flasks for growing the HCECs) and 3 other wells, in which the tissue culture plastic was uncoated.

After 12 h the media was removed and the coverslips were washed 3× with PBS. The cells were fixed by adding 1 ml of 4% formaldehyde and left for 10 minutes at room temperature. The formaldehyde was then removed and cover slips were washed 3× with PBS. The cells were stained with about 550 μl DAPI (1:250)/well and left for 10 minutes. The DAPI was removed and the polymers washed 3× with PBS.

### 3. Analysis:

The cells were counted using a fluorescent microscope and the data analysed. When images were obtained, corneal cells were counted using IMSTAR software.

Nine areas of a single cover slip were scanned using the pathfinder system from IMSTAR (IMSTAR. S.A.)—automated image capture and analysis was carried out using the High Content Screening (HCS) platform to count cells and surface area coverage and then give a percentage coverage evaluation.

### Results

| | Number of | Average | Total | | |
|---|---|---|---|---|---|
| | nuclei per | Nuclei | nuclei | | Std Dev |
| | single square | Area | area | % nuclei | (% Nuclei |
| Copolymer | of cover slip | [I.S.U] | [I.S.U] | coverage | Coverage) |
| Example 1 | 130 | 290 | 37694 | 3.28 | 1.29 |
| Example 4 | 219 | 302 | 67096 | 5.83 | 1.92 |
| Example 5 | 36 | 329 | 11339 | 0.99 | 0.86 |
| Example 6 | 142 | 671 | 107142 | 9.32 | 8.18 |

Number of nuclei per Single Square of cover slip—average from nine squares (cell number).

Average Nuclei Area is not equal to cell area. Cell area is unknown.

[I.S.U]—integral system units—in this particular case 1I.S.U=5.85 μm².

Std Dev (% Nuclei Coverage).

### INDUSTRIAL APPLICABILITY

The copolymers indicated as useful for this invention may be incorporated into corneal bandage structures to provide a substrate with affinity for corneal cells. Thereby the bandage is able to maintain a reservoir of cells which have been applied to the bandage, for use in corneal therapy such as described in the above mentioned papers by Pellegrini et al in the Lancet and by Xiaojie Hu et al in ‘Tissue Engineering’. Suitably, corneal cells are allowed to multiply on the substrate before use of the bandage to ensure an adequate cell population for therapy.

The active copolymers may be formed into fibres and fabricated into scaffolds to support corneal cells as described by Xiaojie Hu et al. Alternatively a suitable scaffold may be prepared by coating the indicated active polymers over fibres of another polymer.

In another alternative approach, active copolymers may be formed into films or into lens shapes for use as a corneal bandages.

More preferably, active copolymers are provided as a layer, for example, by graft polymerisation or by coating over pre-formed contact lens structures made from polymers suitable for contacts lens (e.g. having suitable transparency and oxygen transmission). The layer may be continuous or discontinuous. For example, the bandage may prepared by dip coating a lens structure in solutions of the active polymers, or alternatively using ink jet dispensing of the active polymers. The cells are grown on, or in the presence of, the soft lens using the coating as substrate to retain a suitable cell population on the lens. This is then placed onto the patient's eye and the cells transfer from the soft lens onto the damaged cornea.

1. A method of corneal therapy, comprising:
applying a substrate to a cornea, wherein the substrate includes a surface coated with a copolymer comprising units -(A)- and -(B)- and corneal cells grown thereon with a population suitable for corneal therapy, in which the units A are derived from aminoalkyl (alkyl)acrylates; and the units B are derived from aryloxy, alkoxy or hydroxyalkyl (alkyl)acrylates, or (alkyl)acrylic acids.

2. The method according to claim 1, in which the substrate is a corneal bandage

3. The method according to claim 1, in which the copolymer comprises units A having the general formula (D) 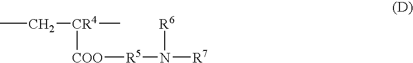
in which
R⁴, R⁶, R⁷ are independently hydrogen, lower alkyl, phenyl or substituted phenyl groups;
R⁵ is CH₂—CH₂, CHR—CH₂, CH₂—CHR^{a}, CHR^{a}—CH₂, CHR^{a}—CHR^{b}, CHR^{a}—CR^{b}R^{c}, CR^{a}R^{b}—CHR^{c}, CR^{a}R^{b}—CR^{c}R^{d}, or (CH₂)ₑ, wherein e is 2, 3, 4, 5, or 6, and R^{a}, R^{b}, R^{c}, R^{d} are lower alkyl groups and R^{a}, R^{b}, R^{c} and R^{d} maybe the same or different.

4. The method according to claim 1,in which the copolymer comprises units B having the general formula (E) 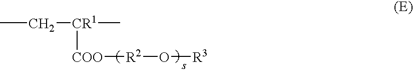
in which
R¹ is H, a lower alkyl, phenyl or substituted phenyl group;
R² is CH₂—CH₂, CH₂—CHR^{a}, CHR^{a}—CH₂, CHR^{a}—CHR^{b}, CHR^{a}—CR^{b}R^{c}, CR^{a}R^{b}—CHR^{c}, CR^{a}R^{b}—CR^{c}R^{d}, or (CH₂)ₑ, wherein e is 2, 3, 4, 5, or 6, and R^{a}, R^{b}, R^{c}, R^{d} are lower alkyl groups and R^{a}, R^{b}, R^{c} and R^{d} maybe the same or different;
s is 0 or 1;
and when s is 0, R³ is hydrogen;
and when s is 1, R³ is hydrogen or a lower alkyl, phenyl or substituted phenyl group.

5. The method according to claim 1, in which the copolymer has the structure
-(A)ₚ-(B)_{q}-
in which p+q=100, and 0<p, q<100.

6. The method according to claim 5, in which the ratio of p:q is in the range of 10-70:90-30.

7. The method according to claim 1, in which in units B, s is 1 and R³ is hydrogen.

8. The method according to claim 1, in which in units B, s is 1 and R³ is a lower alkyl group.

9. The method according to claim 1, in which in units B, s is 0 and R³ is hydrogen.

10. The method according to claim 1, in which unit A of the copolymer is derived from 2-(diethylamino)ethyl methacrylate (DEAEMA), 2-(diethylamino)ethyl acrylate (DEAEA), 2-(dimethylamino)ethyl methacrylate (DMAEMA), or 2-(dimethylamino)ethyl acrylate (DMAEA), or mixtures thereof.

11. The method according to claim 1, in which unit B of the copolymer is derived from hydroxylethylmethacrylate (HEMA), hydroxypropylmethacrylate (HPMA), hydroxylbutylmethacrylate (HBMA), hydroxylethylacrylate (HEA), hydroxylpropylacrylate (HPA), hydroxylbutylacrylate (HBA), or methacrylic acid (MAA), or mixture thereof.

12. The method according to claim 1, in which unit A of the copolymer is derived from 2-(dimethylamino)ethyl methacrylate (DMAEMA) and unit B derived from methacrylic acid (MAA).

13. The method according to claim 1, in which unit A of the copolymer is derived from 2-(diethylamino)ethyl methacrylate or 2-(dimethylamino)ethyl methacrylate (DEAEMA or DMAEMA) and B is derived from a hydroxyl(ethyl or propyl)methacrylate (HEMA or HPMA).

14. The method according to claim 1, in which A of the copolymer is derived from 2-(diethylamino)ethylacrylate (DEAEA) and B is derived from 2-methoxyethylmethacrylate (MEMA).

15. A corneal bandage comprising: a polymeric material in which at least the surface comprises a copolymer comprising units -(A)- and -(B)- in which
the units A are derived from aminoalkyl (alkyl)acrylates; and
the units B are derived from aryloxy, alkoxy or hydroxyalkyl (alkyl)acrylates, or (alkyl)acrylic acids.

16. A corneal bandage according to claim 15, comprising fibres formed from or coated with said copolymer.

17. A corneal bandage according to claim 15, comprising a polymeric contact lens having a surface layer of said copolymer.

18. A corneal bandage, comprising:
a bandage support with at least a surface layer of a copolymer comprising units -(A)- and -(B)- in which
the units A are derived from aminoalkyl (alkyl)acrylates; and
the units B are derived from aryloxy, alkoxy or hydroxyalkyl (alkyl)acrylates or (alkyl)acrylic acids
to provide a surface having an affinity to corneal cells.

19. A corneal bandage according to claim 18 in which the bandage support is a fibrous support.

20. A corneal bandage according to claim 18 in which the bandage support is a contact lens.

21. A corneal bandage according to claim 18 in which the support is coated or imprinted with the copolymer.

22. A method of preparing a corneal bandage for corneal therapy, comprising:
providing a contact lens with at least a surface layer of a copolymer comprising units -(A)- and -(B)- in which
the units A are derived from aminoalkyl (alkyl)acrylates; and
the units B are derived from aryloxy, alkoxy or hydroxyalkyl (alkyl)acrylates or (alkyl)acrylic acids
to provide a surface having an affinity to corneal cells, and
growing corneal cells on the coated surface of the contact lens to obtain a corneal cell population suitable for corneal therapy.

23. A method according to claim 22, in which the contact lens is a preformed contact lens.

24. A method according to claim 23 in which the contact lens is coated or imprinted with the copolymer.

25. A method according to claim 22, in which the corneal cells are selected from the group consisting of corneal epithelial cells, conjunctival squamous cells and conjunctival goblet cells.

26. A method according to claim 1, in which the substrate is a contact lens.

27. A method according to claim 26, in which the contact lens is a preformed contact lens.

28. A method according to claim 27 in which the contact lens is coated or imprinted with the copolymer.

29. A method according to claim 26, in which the corneal cells are selected from the group consisting of corneal epithelial cells, conjunctival squamous cells and conjunctival goblet cells.
